(19) United States
(12) Patent Application Publication
(10) Pub. No.: US 2016/0338936 A1
(43) Pub. Date: Nov. 24, 2016

(54) Latex Barrier

(71) Applicant: Anamarie Seidel, Olympia, WA (US)

(72) Inventor: Anamarie Seidel, Olympia, WA (US)

(21) Appl. No.: 14/718,853

(22) Filed: May 21, 2015

(51) Int. Cl. A61K 8/81; A61Q 3/00; A61Q 3/02; A61K 8/19

(52) U.S. Cl. CPC A61K 8/81; A61K 8/19; A61Q 3/00; A61Q 3/02

(57) ABSTRACT

A latex barrier is provided which may protect surfaces such as a person's skin from contact, penetration, or damage from objects or substances such as skin-dissolving products like cuticle removers. A latex barrier may also be used as a flexible peel-off base coat. A latex barrier may be easy to apply, dry quickly, have a mild ammonia odor, peel off in one piece, and be durable enough to work with nail art applications such as sponging and marbling, for example. A latex barrier may contain aesthetic elements such as glitter.

### FIELD

This disclosure relates generally to a Latex Barrier.

### BACKGROUND

The anatomy of the human nail includes the nail plate, which is the hard part of the nail, and the eponychium, which is the tight band of skin at the base of the nail plate. The proximal fold of the eponychium serves as a guardian seal to prevent germs and bacteria from entering the matrix, which is the tissue that the nail protects. The cuticle comprises the thin layer of dead cells that grips the underside of the eponychium and attaches to the surface of the nail plate. The cuticle forms a seal between the nail plate and the eponychium to prevent pathogens from infecting the matrix.

Many conventional cuticle remover products contain harmful ingredients such as lye, which may dissolve not only dead cells such as the cuticle, but also important living protein cells such as those of the eponychium. Conventional cuticle removers often cause skin irritation and damage such as hangnails, for example. Meanwhile, conventional liquid latex products are often too thin or take too long to dry. They also emit a strong ammonia odor and may lack durability for nail art applications such as sponging and marbling.

### SUMMARY

The following presents a simplified summary of the disclosure to provide a basic understanding to the reader. This summary is not an extensive overview of the disclosure, nor does it identify key or critical elements of the claimed subject matter or define its scope. Its sole purpose is to present some concepts disclosed in a simplified form as a precursor to the more detailed description that is later presented.

The instant application discloses, among other things, a Latex Barrier, which may protect surfaces, such as a person's skin, from objects or substances such as skin-dissolving cuticle remover products, for example. Latex Barrier may also be used as a flexible peel-off base coat for applications such as nail art. Latex Barrier may comprise a liquid solution that is easy to apply, dries quickly, and peels off in one piece. It may have a mild ammonia odor and may contain aesthetic elements such as glitter, for example. A person skilled in the art will understand that Latex Barrier may comprise various colors and other functional and aesthetic characteristics. Latex Barrier may be used in various contexts including but not limited to nail care and nail art applications, for example.

Many of the attendant features may be more readily appreciated as they become better understood by reference to the following detailed description considered in connection with the attached drawings.

### BRIEF DESCRIPTION OF THE DRAWINGS

FIG. 1 illustrates an anatomy of a human nail.

FIG. 2 illustrates a Latex Barrier composition, according to one embodiment.

FIG. 3 illustrates a Latex Barrier application method, according to one embodiment.

FIG. 4 illustrates a Latex Barrier application method, according to one embodiment.

FIG. 5 illustrates a Latex Barrier applied on a human finger or toe, according to one embodiment.

FIG. 6 illustrates a Latex Barrier product, according to one embodiment.

FIG. 7 illustrates a Latex Barrier product, according to one embodiment.

Like reference numerals are used to designate like parts in the accompanying drawings.

### DETAILED DESCRIPTION

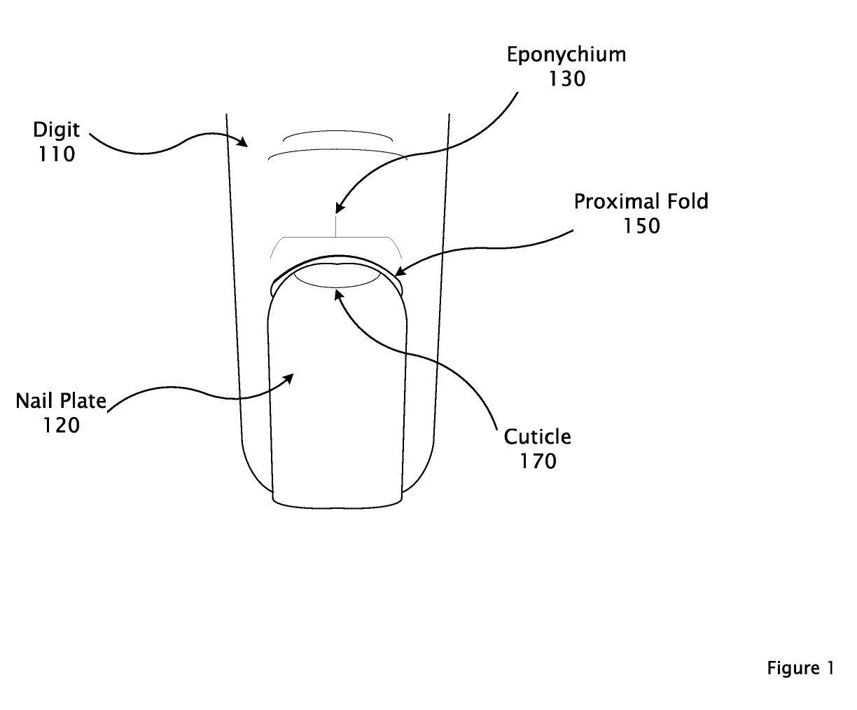

FIG. 1 illustrates an anatomy of a human nail. The human nail may be part of a person's Digit 110. In this example, the human nail may include Nail Plate 120, which may comprise the hard part of the nail. Eponychium 130 may comprise the living skin that covers approximately 20 percent of the Nail Plate 120. Eponychium 130 may serve as a guardian seal which prevents germs and bacteria from entering the tissue that the nail protects. Proximal Fold 150 of Eponychium 130 may comprise the tight band of skin at the base of the Nail Plate 120.

Cuticle 170 may comprise the thin layer of dead cells that grips the underside of Eponychium 130 and attaches to Nail Plate 120. Cuticle 170 may form a seal between Nail Plate 120 and Eponychium 130.

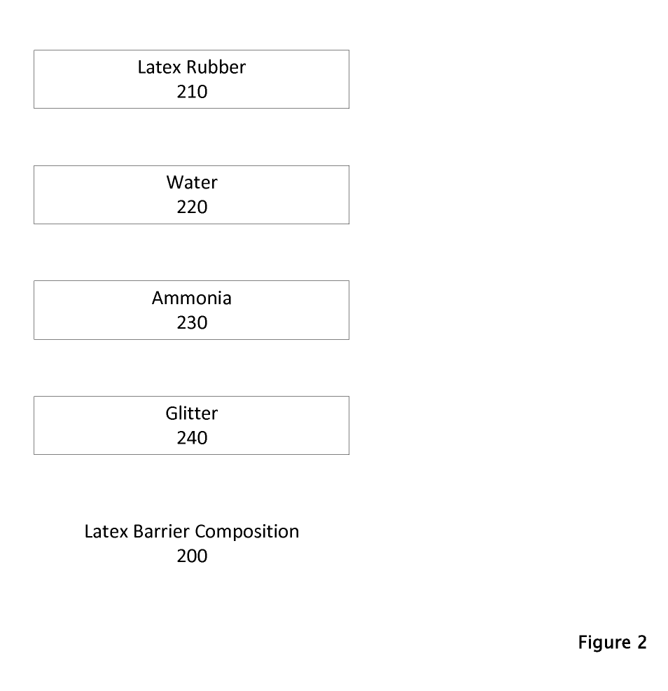

FIG. 2 illustrates a Latex Barrier composition, according to one embodiment. Latex Barrier may protect the skin from contact, irritation, penetration, damage, or other effects, for example, of any objects or substances. In this example, Latex Barrier may comprise a solution which may protect surfaces, such as a person's skin, from skin-dissolving cuticle remover products, for example. Latex Barrier may also protect skin from messes made during nail art applications such as sponging and marbling, for example, facilitating cleanup. Latex Barrier may also be used as a stretchy, flexible peel-off base coat which may last for about 12 hours or any other duration.

In one embodiment, such as in nail care or nail art applications, a user may use an applicator brush to apply Latex Barrier to the skin surrounding a fingernail or toenail, for example. Latex Barrier may comprise a liquid solution which coagulates or dries upon exposure to air. Coagulation or drying may occur after any duration of time, for example, several seconds or minutes. Latex Barrier may have at least one color when it is in liquid form and may change colors upon coagulation or drying. For example, Latex Barrier may display a white color upon application as a liquid and may become translucent upon coagulation or drying. In one embodiment, Latex Barrier may reveal aesthetic elements such as glitter or other embellishments, for example, upon coagulation or drying. The appearance of the aesthetic elements may serve as a signal that Latex Barrier has dried. Upon coagulation or drying, Latex Barrier may become a stretchy, flexible material which a user may remove by peeling, and may be durable enough to be peeled off in one piece. A person skilled in the art will understand that Latex Barrier may have various colors and other functional and aesthetic characteristics. Latex Barrier may be used in various contexts including but not limited to nail care and nail art applications, costume and makeup applications, medical procedures, athletic and recreational activities, and arts and crafts such as painting, woodwork, glasswork, and ceramics, for example. Latex Barrier may have a mild ammonia odor or any other scents.

According to one embodiment, a Latex Barrier composition may comprise Latex Rubber 210, Water 220, a preservative such as Ammonia 230, and at least one aesthetic element such as Glitter 240, for example. In one embodiment, a Latex Barrier composition may combine the following ingredients: approximately 60 ounces (plus or minus approximately one ounce, for example) of a water-based suspension of latex; approximately 60 ounces (plus or minus approximately one ounce, for example) of a natural rubber adhesive; and approximately 150 grams (plus or minus approximately five grams, for example) of an aesthetic element.

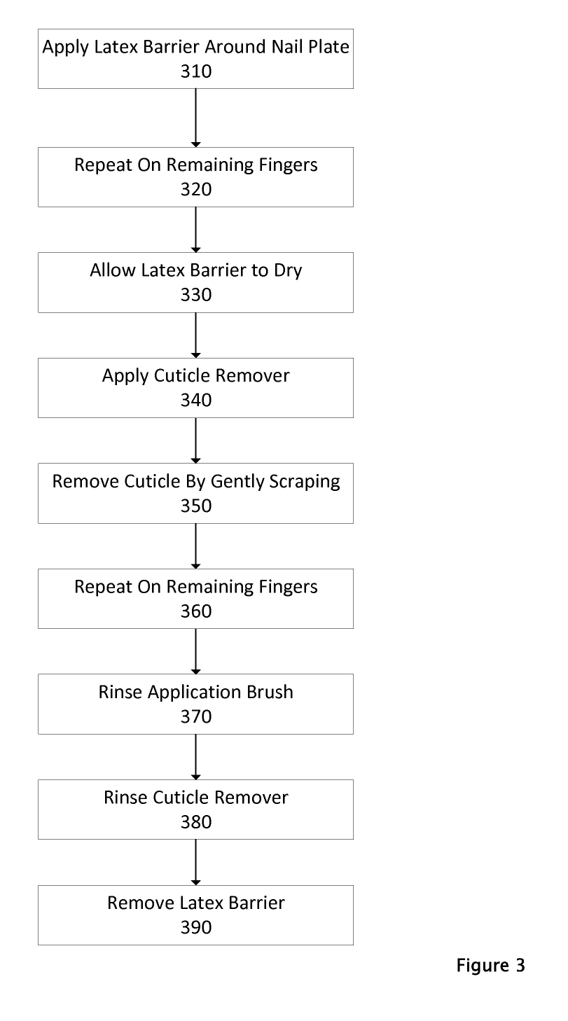

FIG. 3 illustrates a Latex Barrier application method, according to one embodiment. In this example, Latex Barrier may be applied during routine nail care, such as cuticle removal, and the application method may comprise several steps. At Apply Latex Barrier Around Nail Plate 310, a user may apply a thin layer of Latex Barrier on a person's skin surrounding the nail plate. The user may start with the little finger, starting at the midpoint of the cuticle line, pulling to one side, then repeating on the other side of the finger. At Repeat On Remaining Fingers 320, the user may repeat the step of Apply Latex Barrier Around Nail Plate 310 to the remaining nine fingers. If the user gets Latex Barrier on the nail plate, the user may use a device such as an orange wood stick, for example, to remove Latex Barrier before it dries. At Allow Latex Barrier to Dry 330, the user may wait approximately 90 to 120 seconds, for example, or any other duration until Latex Barrier dries. Latex Barrier may change appearance upon coagulation or drying; for example, it may change from displaying a white color to becoming translucent and may expose aesthetic elements such as sparkles from glitter contained in Latex Barrier, for example. The user may expose the Latex Barrier surface to blowing air, for example, to accelerate coagulation or drying.

At Apply Cuticle Remover 340, the user may apply a substance, such as a cuticle remover product, for example to the nail plate, following the manufacturer's directions. In one example, the cuticle remover may turn Latex Barrier a different color, such as white, for example, or any other color. Latex Barrier may prevent the cuticle remover from contacting, penetrating, harming, or otherwise affecting the skin. At Remove Cuticle By Gently Scraping 350, the user may gently slide a cuticle remover tool across the nail plate to loosen the cuticle cells. The user may avoid applying downward pressure with the tool to prevent injury. At Repeat On Remaining Fingers 360, the user may repeat steps 330, 340, and 350 on the remaining nine fingers. At Rinse Application Brush 370, the user may immediately rinse the brush in water before sealing the Latex Barrier container. At Rinse Cuticle Remover 380, the user may wash off the cuticle remover. At Remove Latex Barrier 390, the user may rub the edge of Latex Barrier by using a finger to peel it off. A person skilled in the art will understand that the aforementioned steps may also be applied to a person's toes and other application contexts.

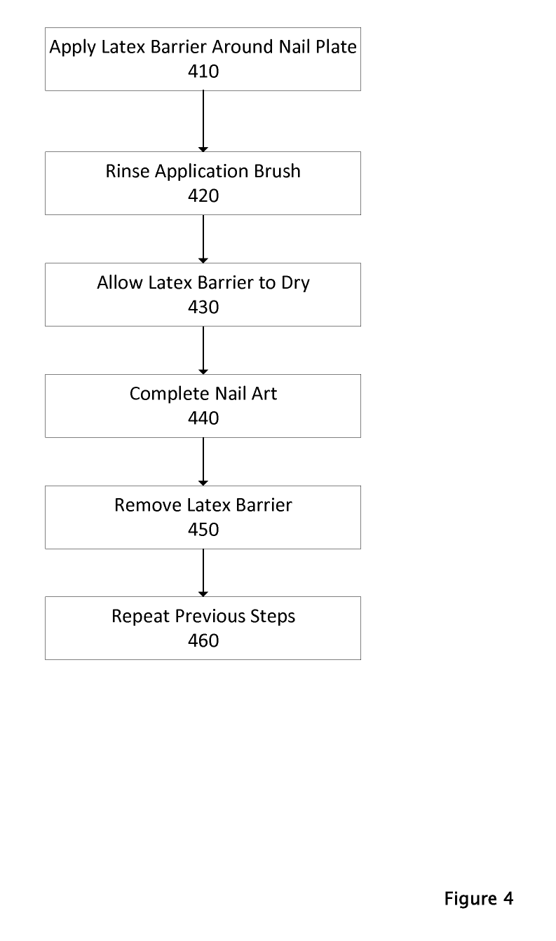

FIG. 4 illustrates a Latex Barrier application method, according to one embodiment. In this example, a Latex Barrier application method with nail art may comprise several steps. At Apply Latex Barrier Around Nail Plate 410, a user may apply a thin layer around the nail plate of every other finger, for example. The Latex Barrier may stick to itself. At Rinse Application Brush 420, the user may immediately rinse the brush in water before sealing the Latex Barrier bottle. At Allow Latex Barrier to Dry 430, the user may allow Latex Barrier to coagulate or dry for about 90 to 120 seconds or until the Latex Barrier surface sparkles, for example. The user may expose the Latex Barrier surface to blowing air or UV lighting, for example, to speed accelerate coagulation or drying. At Complete Nail Art 440, the user may complete nail art such as stamping, marbling, sponging, or gradient designs. The user may use non-latex sponges for gradient designs, for example. Latex Barrier may protect the skin from skin damage or messes made during the nail art application, facilitating cleanup. Latex At Remove Latex Barrier 450, user may rub an edge of Latex Barrier with a finger and peel to remove it. At Repeat Previous Steps 460, the user may repeat Steps 410 through 450 with the remaining nails. A person skilled in the art will understand that the aforementioned steps may also be applied to a person's toes and other application contexts.

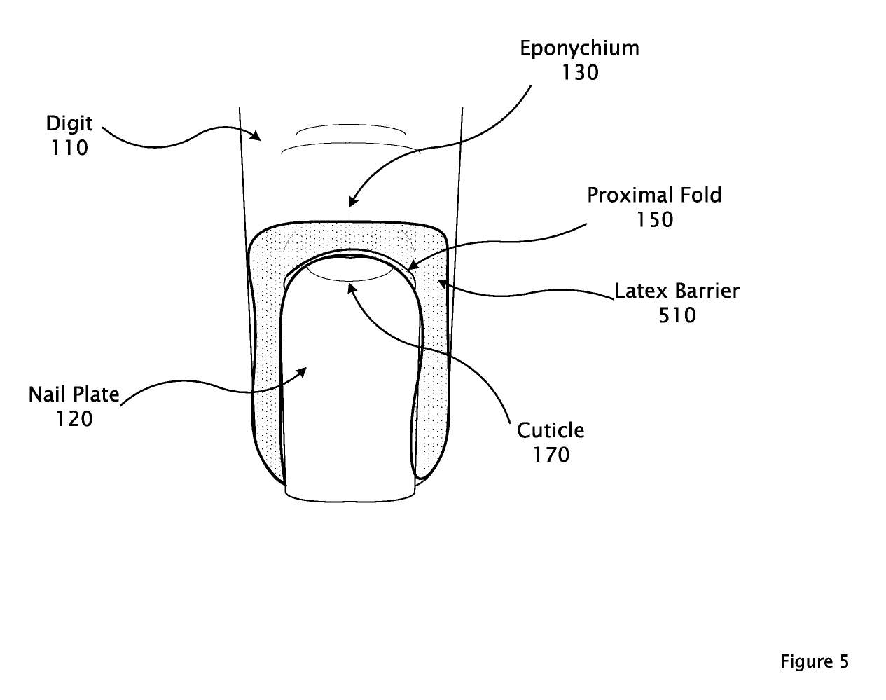

FIG. 5 illustrates a Latex Barrier applied on a human finger or toe, according to one embodiment. In this example, Latex Barrier 510 may comprise a liquid latex solution which, when dried, may serve as a flexible and peelable protective base coat to prevent contact, penetration, damage, and other effects to skin surrounding the nail during nail art or nail care applications such as cuticle removal, for example. Latex Barrier 510 may change characteristics upon coagulation or drying; for example, it may change colors or translucency and may display aesthetic elements such as Glitter 520 upon coagulation or drying.

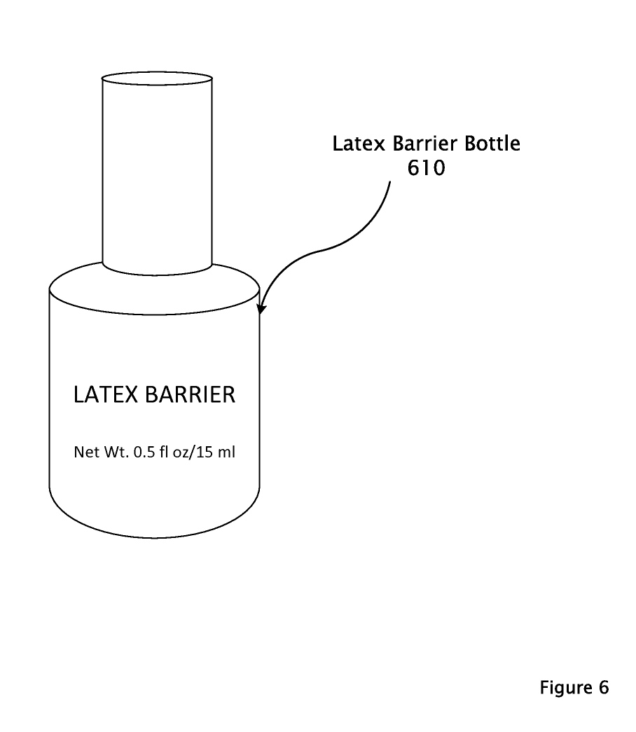

FIG. 6 illustrates a Latex Barrier product, according to one embodiment. In this example, Latex Barrier may be contained in a Latex Barrier Bottle 610, for example, a 0.5-ounce starter bottle with an application brush, or any other packaging format. The application brush may be made of plastic, which may facilitate cleaning, as liquid latex may destroy natural or synthetic hair art brushes. In another embodiment, Latex Barrier Bottle 610 may include an applicator that uses air to release the Latex Barrier solution in droplets, or it may have an applicator that releases it by squirting, spraying, or rolling onto a surface, for example.

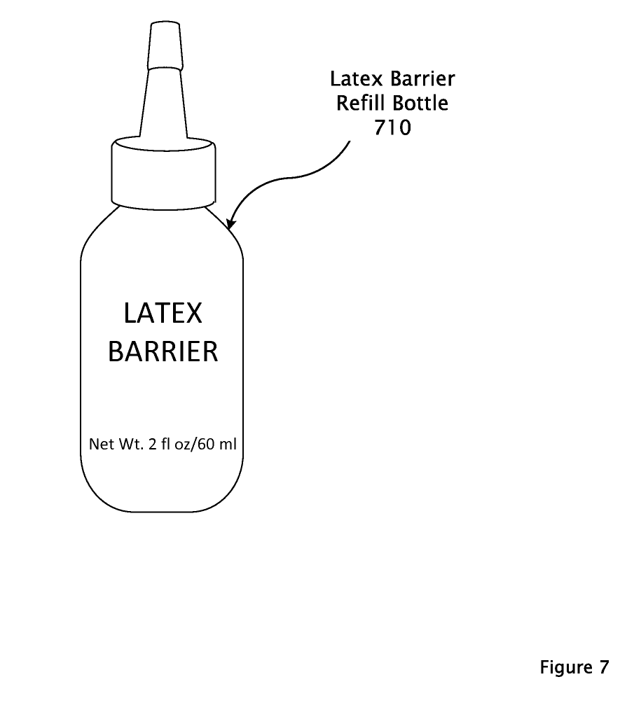

FIG. 7 illustrates a Latex Barrier product, according to one embodiment. In this example, Latex Barrier Refill Bottle 710 may be contained in a 2-ounce refill bottle, or any other packaging format.

While the detailed description above has been expressed in terms of specific examples, those skilled in the art will appreciate that many other configurations could be used. Accordingly, it will be appreciated that various equivalent modifications of the above-described embodiments may be made without departing from the spirit and scope of the invention.

Additionally, the illustrated operations in the description show certain events occurring in a certain order. In alternative embodiments, certain operations may be performed in a different order, modified or removed. Moreover, steps may be added to the above-described logic and still conform to the described embodiments. Further, operations described herein may occur sequentially, or certain operations may be processed in parallel.

The foregoing description of various embodiments of the invention has been presented for the purposes of illustration and description. It is not intended to be exhaustive or to limit the invention to the precise form disclosed. It is intended that the scope of the invention be limited not by this detailed description, but rather by the claims appended hereto. The above specification, examples, and data provide a complete description of the manufacture and use of the invention. Since many embodiments of the invention can be made without departing from the spirit and scope of the invention, the invention resides in the claims hereinafter appended.

1. A latex barrier, comprising:
a solution comprising latex rubber, water, at least one preservative, and at least one aesthetic element, the solution configured to serve as a protective barrier for human skin.

2. The solution of claim 1, wherein the preservative is ammonia.

3. The solution of claim 1, wherein the aesthetic element is glitter.

4. The solution of claim 1, further configured to function as a protective base coat.

5. The solution of claim 1, wherein the solution is flexible.

6. The solution of claim 1, wherein the solution is peelable.

7. The solution of claim 1, wherein the solution changes color upon drying.

8. The solution of claim 1, wherein the solution composition comprises approximately 60 ounces of a water-based suspension of latex; approximately 60 ounces of a natural rubber adhesive; and approximately 150 grams of an aesthetic element.
